(19) United States
(12) Patent Application Publication
(10) Pub. No.: US 2013/0110169 A1
(43) Pub. Date: May 2, 2013

(54) VERTEBRAL ROD SYSTEM AND METHODS OF USE

(71) Applicants: Richard A. Hynes, Melbourne Beach, FL (US); Todd Lanman, Palos Verdes Estates, CA (US); Julien Prevost, Memphis, TN (US); Christopher Scifert, Bartlett, TN (US)

(72) Inventors: Richard A. Hynes, Melbourne Beach, FL (US); Todd Lanman, Palos Verdes Estates, CA (US); Julien Prevost, Memphis, TN (US); Christopher Scifert, Bartlett, TN (US)

(73) Assignee: WARSAW ORTHOPEDIC, INC., Warsaw, IN (US)

(21) Appl. No.: 13/282,266

(22) Filed: Oct. 26, 2011

(51) Int. Cl. A61B 17/70

(52) U.S. Cl. 606/254

(57) ABSTRACT

A vertebral rod comprises an elongated first section and an elongated second section. A flexible intermediate section is disposed between the first section and the second section. The intermediate section has an interior surface defining a cavity having an open end. A resistance member is disposed to occupy the entire cavity of the intermediate section. The resistance member includes an outer surface such that the entire interior surface engages the outer surface during movement of the intermediate section. Methods of use are disclosed.

### TECHNICAL FIELD

The present disclosure generally relates to medical devices for the treatment of spinal disorders, and more particularly to a dynamic vertebral rod system, having flexion and extension capability, which provides stability while reducing stress on spinal elements.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes discectomy, laminectomy, fusion and implantable prosthetics. As part of these surgical treatments, connecting elements such as vertebral rods are often used to provide stability to a treated region. During surgical treatment, one or more rods may be fastened to the exterior of two or more vertebral members. For example, such vertebral rods can be fastened using screws or similar type fasteners. This disclosure describes an improvement over these prior art technologies.

### SUMMARY OF THE INVENTION

Accordingly, a dynamic vertebral rod system is provided, having flexion and extension capability, which provides stability while reducing stress on spinal elements. It is envisioned that the disclosed system may be employed as a posterior, anterior and/or lateral dynamic stabilization device. The components of the vertebral rod system are easily manufactured and assembled.

In one embodiment, a vertebral rod comprises an elongated first section and an elongated second section. A flexible intermediate section is disposed between the first section and the second section. The intermediate section has an interior surface defining a cavity having an open end. A resistance member is disposed to occupy the entire cavity of the intermediate section. The resistance member includes an outer surface such that the entire interior surface engages the outer surface during movement of the intermediate section.

In one embodiment, the vertebral rod system includes an intermediate section having an at least partially circumferential interior surface defining a cavity having an open end. The intermediate section includes a first elongated recess within the interior surface disposed adjacent the first section and a second opposing elongated recess within the interior recess disposed adjacent the second section. A resistance member is disposed in the cavity of the intermediate section and has an at least partially circumferential outer surface. The resistance member includes a first elongated protrusion extending along the outer surface and a second elongated protrusion extending along the outer surface. The first protrusion is disposed in the first recess and the second protrusion is disposed in the second recess such that the entire interior surface engages the entire outer surface.

In one embodiment, an implant system is provided, which includes a vertebral rod comprising a first section, a second section configured to extending along a plurality of vertebral levels and an intermediate section disposed between the first section and the second section. The intermediate section has an interior surface defining a cavity having an open end. The intermediate section further has at least one elongated recess within the interior surface. A resistance member is disposed in the cavity of the intermediate section. The resistance member includes an outer surface having at least one elongated protrusion extending therealong. The at least one protrusion is disposed in the at least one recess such that the entire interior surface engages the outer surface. A first bone fastener is provided for attaching the first section to tissue. A second bone fastener is provided for attaching the second section to tissue.

### BRIEF DESCRIPTION OF THE DRAWINGS

The present disclosure will become more readily apparent from the specific description accompanied by the following drawings, in which:

FIG. 1 is a perspective one embodiment of a rod of a system in accordance with the principles of the present disclosure;

FIG. 2 is a side view of the rod shown in FIG. 1;

FIG. 3 is a perspective view of a resistance member of the system in accordance with the principles of the present disclosure;

FIG. 4 is a perspective view of the rod and the resistance member of the system in accordance with the principles of the present disclosure;

FIG. 5 is a perspective view of the system in accordance with the principles of the present disclosure attached with vertebrae;

FIG. 6 is side view, in part cross section, of the system attached with vertebrae shown in FIG. 5; and

FIGS. 7 and 8 are plan views of the rod shown FIG. 6 in flexion and extension, respectively.

Like reference numerals indicate similar parts throughout the figures.

### DETAILED DESCRIPTION OF THE INVENTION

The exemplary embodiments of the vertebral rod system and methods of use disclosed are discussed in terms of medical devices for the treatment of spinal disorders and more particularly, in terms of a dynamic vertebral rod system having flexion and extension capability.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed surgical system and methods may be alternatively employed in a surgical treatment with a patient in a prone or supine position, and/or employ various surgical approaches to the spine, including anterior, posterior, posterior mid-line, lateral, posterolateral, and/or anterolateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drags to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

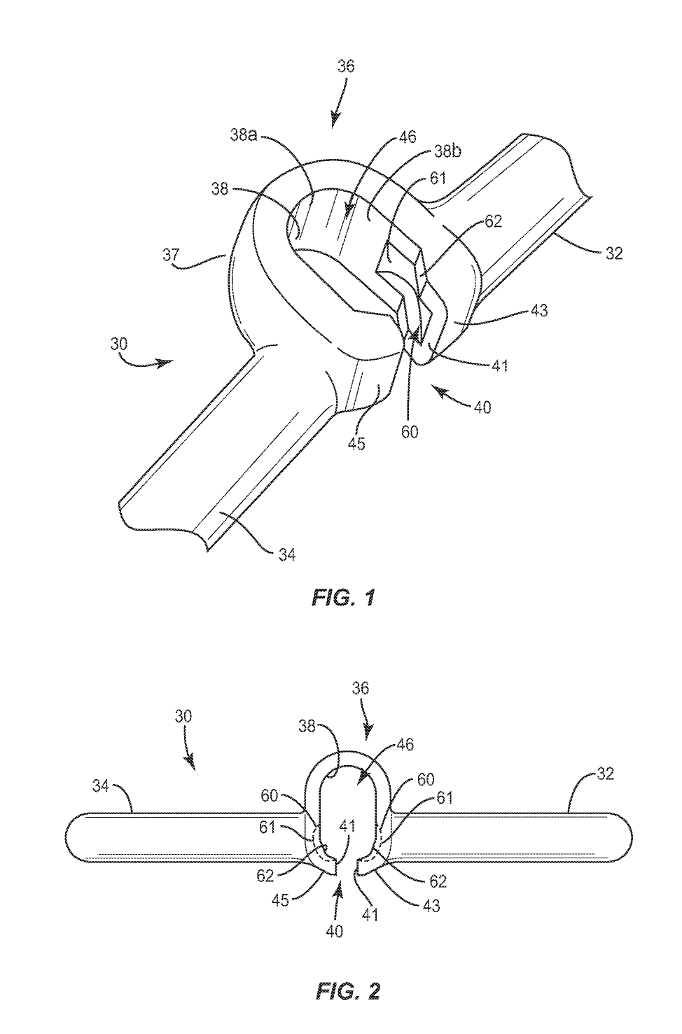

The following discussion includes a description of a vertebral rod system, related components and exemplary methods of employing the vertebral rod system in accordance with the principles of the present disclosure. Alternate embodiments are also disclosed. Reference will now be made in detail to the exemplary embodiments of the present disclosure, which are illustrated in the accompanying figures. Turning now to FIGS. 1-4, there is illustrated components of a vertebral rod system in accordance with the principles of the present disclosure.

The components of the vertebral rod system can be fabricated from biologically acceptable materials suitable for medical applications, including metals, synthetic polymers, ceramics and bone material and/or their composites, depending on the particular application and/or preference of a medical practitioner. For example, the components of the system, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of the system may have material composites, including the above materials, to achieve various desired characteristics such as strength, rigidity, elasticity, compliance, biomechanical performance, durability and radiolucency or imaging preference. The components of the system, individually or collectively, may also be fabricated from a heterogeneous material such as a combination of two or more of the above-described materials. The components of the system may be monolithically formed, integrally connected or include fastening elements and/or instruments, as described herein.

For example, the vertebral rod can be formed of two or more materials. In one embodiment, elongated rod sections can be fabricated from carbon-reinforced PEEK and an intermediate section can be fabricated from PEEK. In another embodiment, elongated rod sections are fabricated from PEEK and an intermediate section is fabricated from carbon-reinforced PEEK. In another embodiment, alternate materials may be employed in a radial direction of a vertebral rod such that stiff materials such as metals or other composites are used in a core of the rod sections and an outer sheet of lower modulus polymeric material is used in the outer radial portion of the rod sections, or vice versa. In another embodiment employing a composite material similar to those described, the elongated rod sections can have a cylindrical geometry and the intermediate section can have a rectangular or oblong geometry.

As a further example, a resistance member of the vertebral rod system may be fabricated from materials such as silicone, polyurethane, silicone-polyurethane copolymers, polymeric rubbers, polyolefin rubbers, hydrogels, semi-rigid and rigid materials, and biocompatible materials such as elastomers, rubbers, thermoplastic elastomers, thermoset elastomers, elastomeric composites and plastics. It is envisioned that the rod sections can be manufactured from, for example, machining and milling from a solid stock material and/or injection molding. The resistance member can be manufactured from, for example, machining and milling, extrusion and die cutting, injection molding, transfer molding and/or cast molding. One skilled in the art, however, will realize that such materials and fabrication methods suitable for assembly and manufacture, in accordance with the present disclosure, would be appropriate.

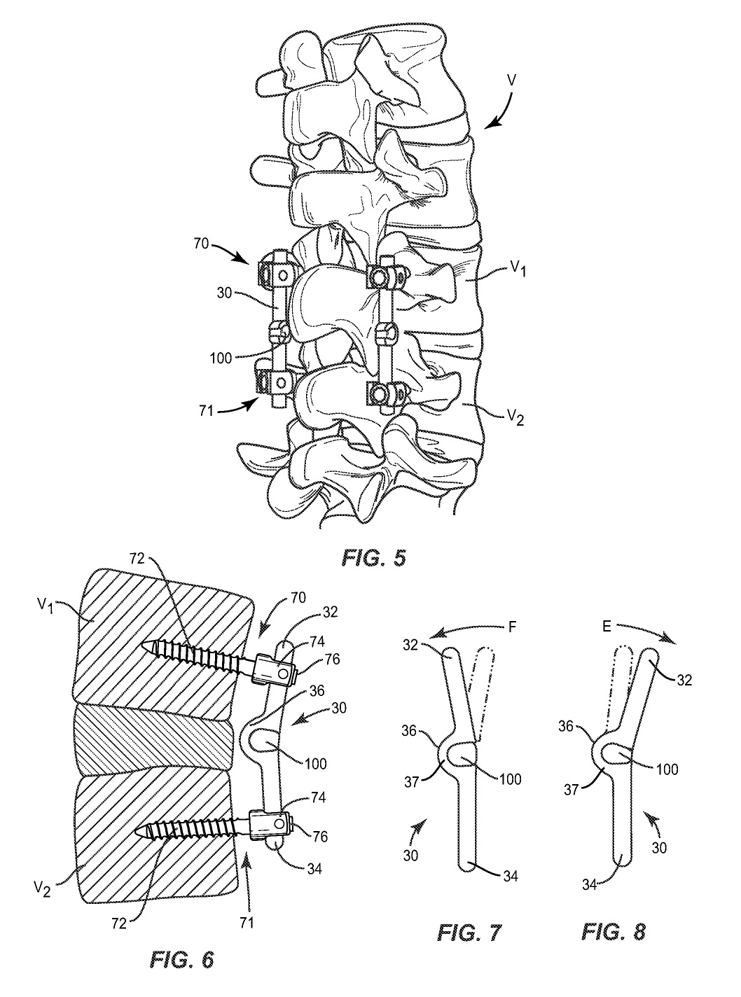

Referring to FIGS. 1 and 2, the vertebral rod system is configured for attachment to vertebrae (as shown, for example, in FIG. 5) during surgical treatment of a spinal disorder, examples of which are discussed herein. The vertebral rod system has a vertebral rod 30, which includes a first elongated section, such as, for example, upper section 32 and a second elongated section, such as, for example, lower section 34.

An intermediate section 36 is connected with sections 32, 34 and disposed therebetween as a joining section of the components of vertebral rod 30. It is envisioned that the components of vertebral rod 30 may be monolithically formed, integrally connected or arranged with attaching elements. Intermediate section 36 is flexible relative to sections 32, 34, and is configured to provide resistance to movement of sections 32, 34. It is envisioned that intermediate section 36 may provide increasing, variable, constant and/or decreasing resistance. It is contemplated that sections 32, 34, 36 can be variously dimensioned, for example, with regard to length, width, diameter and thickness. It is further contemplated that the respective cross-section of sections 32, 34, 36 may have various configurations, for example, round, oval, rectangular, irregular, uniform and non-uniform. Section 32 may have a different cross-sectional area, geometry, material or material property such as strength, modulus or flexibility relative to section 34.

Intermediate section 36 may have a variable thickness according to the requirements of the particular application. It is envisioned that thickness of intermediate section 36 may be in a range of 1-15 mm, preferably in a range of 2-8 mm, and most preferably in a range of 3-5 mm. It is further envisioned that the cross-sectional geometry or area of intermediate section 36 can be uniform, non-uniform, consistent or variable.

It is envisioned that intermediate section 36 may be configured as a flexible joint having a wide, narrow, round or irregular configuration. It is further envisioned that intermediate section 36 can be variously configured and dimensioned with regard to size, shape, thickness, geometry and material. Intermediate section 36 may be fabricated from the same or alternative material to sections 32, 34. Intermediate section 36 may also have a different cross-sectional area, geometry or material property such as strength, modulus and flexibility relative to sections 32, 34. Intermediate section 36 may be connected to sections 32, 34 using various methods and structure including molding of a continuous component, mechanical fastening, adhesive bonding and combinations thereof.

It is envisioned that intermediate section 36 has a flexible hinge configuration, which can be offset forward or backward relative to a central axis of rod 30 to modify the flexibility or stiffness of the vertebral rod system. It is further envisioned that particular parameters may be selected to modulate the flexibility or stiffness of the vertebral rod system including the cross-sectional area (or thickness) of intermediate section 36, material modulus that may correlate to the hardness of bumper 100 discussed below, modification of porosity in a range of 0-30 percent which may include modification of void volume in a range of 10 microns-1 mm, as well as rod material properties. These parameters allow modification of the properties or performance of the vertebral rod system such as strength, durability, flexibility (or stiffness), overall profile and the ability to employ a percutaneous approach, for a particular application.

Intermediate section 36 includes a flexible joint member 37, which has a C-shaped configuration and defines a corresponding shaped arcuate inner surface 38 having a rear portion 38a, opposite side portions 38b, and an open end 40 defined by opposing planar surfaces 41. It is contemplated that joint member 37 may have alternative configurations such as U-shaped, V-shaped or W-shaped. It is further contemplated that vertebral rod 30 may include one or a plurality of intermediate sections 36 spaced along the length of rod 30. In embodiments including a plurality of sections 36, the multiple sections 36 may be disposed in similar, or alternative orientations such as aligned, non-aligned, offset, open end facing or not facing vertebrae and alternate angular orientation.

Upper section 32 is disposed adjacent to an upper portion of open end 40 and the transition defines a front face 43. Lower section 34 is disposed adjacent a lower portion and the transition defines a front face 45. Inner surface 38 defines a cavity 46, which has a first distance and a second distance adjacent the open end 40, the first distance being greater than the second distance so as to configure a narrowed open end 40. Inner surface 38 further defines two opposing recesses 60, each having an arcuate surface 61 bordered by inclined surface 62.

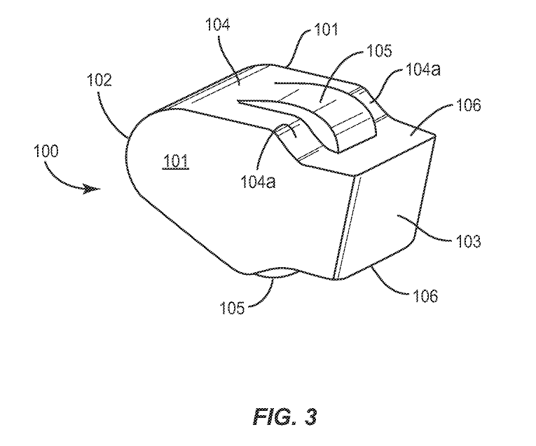

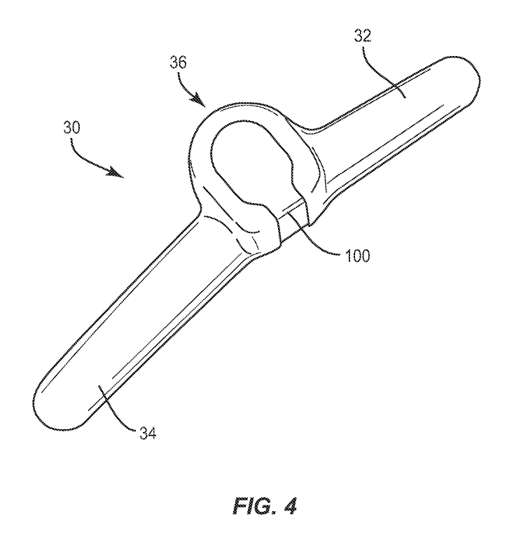

Referring also now to FIGS. 3 and 4, cavity 46 is configured for disposal within of a resistance member, such as, for example, a bumper 100, as shown in detail in FIG. 3. The outer surface of bumper 100 includes opposite side surface portions 101, an arcuate rear surface portion 102, a front surface 103, top and bottom surface portions 104, each having an inclined portion 104a, a protrusion 105 on each of the top and bottom surface portions, and oppositely facing planar surfaces, i.e., flanges 106. When inserted into cavity 46 the arcuate rear surface portion 102 abuts the rear portion 38a of the arcuate inner surface 38; the top and bottom surfaces 104 abut the side surfaces 38b of the inner surface 38 with protrusions 105 each being disposed within a respective one of recesses 60 and abutting the arcuate surface 61 of the respective recess. The inclined surface portion 104a of the top and bottom surface portions 104 abut the inclined surface 62. Flanges 106 abut the opposing surfaces 41. The front surface portion 103 faces outward from the open end 40 and can be flush with the front faces 43 and 45 of the vertebral rod 30. In one embodiment, the bumper 100 can extend beyond the cavity 46 of the intermediate section 36 in a bulging, overflowing and/or overfilled configuration. It is contemplated that bumper 100 is disposed to occupy the entire cavity 46 during movement, such as, for example, expansion and/or compression of intermediate section 36, as described. It is further contemplated that this configuration prevents tissue ingrowth in cavity 46.

Bumper 100 is elastic and configured to provide variable resistance to movement of sections 32, 34 and 36. It is contemplated that bumper 100 can provide increasing, variable, constant and/or decreasing resistance. Bumper 100 is disposed within cavity 46 and engages surface 38 in a close fitting engagement. Bumper 100 can be variously configured with regard to size, shape, for example, round, oblong, rectangular, triangular, spherical, and irregular shapes, and can be of monolithic construction. It is envisioned that bumper 100 has a hardness in the range of 20 Shore A to 55 Shore D, and preferably between 70 and 90 Shore A. The material of bumper 100 can be solid or porous, homogeneous or heterogeneous, single polymer or a blend/composite of more than one polymer. It is contemplated that the resiliency of bumper 100 can prevent creep and improve shape recovery of the vertebral rod system. It is envisioned that bumper 100 is configured to prevent and/or resist closing of open end 40. It is further envisioned that bumper 100 is secured in place with intermediate section 36, and desirably mechanically secured therewith in a configuration to present migration and expulsion therefrom. In other embodiments, bumper 100 can be textured, encapsulated, adhesively bonded and/or over molded with vertebral rod 30. Bumper 100 can be inserted with cavity 46 for assembly, or formed in situ by, for example, a pouch, bag or balloon with the bumper configuration being inserted into cavity 46 and injected with a curable material.

In assembly, operation and use, the vertebral rod system is employed with a surgical procedure for treatment of a spinal disorder affecting a section of a spine of a patient, as discussed herein. The vertebral rod system may also be employed with other surgical procedures. In particular, the vertebral rod system is employed with a surgical procedure for treatment of a condition or injury of an affected section of the spine including vertebrae V, as shown in FIGS. 5 and 6. It is contemplated that the vertebral rod system is attached to vertebrae V for dynamic stabilization of the affected section of the spine to facilitate healing and therapeutic treatment, while providing flexion and extension capability.

In use, to treat the affected section of the spine, a medical practitioner obtains access to a surgical site including vertebra V in any appropriate manner, such as through incision and retraction of tissues. It is envisioned that the vertebral rod system may be used in any existing surgical method or technique including open surgery, mini-open surgery, minimally invasive surgery and percutaneous surgical implantation, whereby the vertebrae V is accessed through a micro-incision, or sleeve that provides a protected passageway to the area. Once access to the surgical site is obtained, the particular surgical procedure is performed for treating the spinal disorder. The vertebral rod system is then employed to augment the surgical treatment. The vertebral rod system can be delivered or implanted as a pre-assembled device or can be assembled in situ. The vertebral rod system may be completely or partially revised, removed or replaced, for example, replacing bumper 100 only, replacing rod 30 and bumper 100 and using the in-place fastening elements.

Referring to FIGS. 5 and 6, a first fastening element, such as, for example, fixation screw assembly 70 is configured to attach upper section 32 to vertebra V₁. A second fastening element, such as, for example, fixation screw assembly 71 is configured to attach lower section 34 to adjacent vertebra V₂. Pilot holes are made in vertebrae V₁, V₂ for receiving fixation screw assemblies 70, 71. Fixation screw assemblies 70, 71 include threaded bone engaging portions 72 that are inserted or otherwise connected to vertebrae V₁, V₂, according to the particular requirements of the surgical treatment. Fixation screw assemblies 70, 71 each have a head 74 with a bore, or through opening and a setscrew 76, which is torqued on to sections 32, 34 to attach rod 30 in place with vertebrae V, as will be described.

As shown in FIG. 5, the vertebral rod system includes two axially aligned and spaced rods 30, with portions of sections 32, 34 extending through the bores of heads 74. Setscrews 76 of each head 74 are torqued on the end portions of rods 30 to securely attach rods 30 with vertebrae V₁, V₂. Upon fixation of the vertebral rod system with vertebrae V, vertebral rod 30 is configured to provide increasing resistance to movement of sections 32, 34 during flexion and extension of the spine. For example, vertebral rod 30, as shown in FIG. 6, is in an unloaded state, which corresponds to the first orientation discussed above, where there is no appreciable tensile or compressive loads on vertebrae V₁, V₂. In flexion and/or extension of vertebrae V caused by corresponding movement of the patient, rod 30 reacts with increasing resistance during movement of rod 30 to a second, third or more orientation(s).

In flexion, as shown in FIG. 7, upper section 32 moves relative to section 34, in the direction of arrow F. Joint member 37 flexibly compresses circumferentially about bumper 100. This configuration increases resistance during flexion. In extension, as shown in FIG. 8, upper section 32 moves relative to section 34, in the direction shown by arrow E. Joint member 37 flexibly expands circumferentially about bumper 100 such that intermediate section 36 compresses bumper 100. Inner surface 38 adjacent bumper 100 is in tension. Resistance is increased during extension. The increase of resistance during flexion and extension provides limited movement of vertebrae V for dynamic stabilization of the treated area of the spine.

The vertebral rod system can be used with various bone screws, pedicle screws or multi-axial screws (MAS) used in spinal surgery. It is contemplated that the vertebral rod system may be used with pedicle screws coated with an osteoinductive material such as hydroxyapatite and/or osteoinductive agent such as a bone morphogenic protein for enhanced bony fixation to facilitate motion of the treated spinal area. Rod 30 and bumper 100 can be made of radio lucent materials such as polymers. Radiomarkers may be included for identification under x-ray, fluoroscopy, CT or other imaging techniques. Metallic or ceramic radiomarkers, such as tantalum beads, tantalum pins, titanium pins, titanium endcaps and platinum wires can be used, such as being disposed at the end portions of rod 30 and/or along the length thereof adjacent joint member 37 or with bumper 100.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A vertebral rod comprising:
an elongated first section;
an elongated second section;
a flexible intermediate section disposed between the first section and the second section, the intermediate section having an interior surface defining a cavity having an open end; and
a resistance member disposed to occupy the entire cavity of the intermediate section, the resistance member including an outer surface such that the entire interior surface engages the outer surface during movement of the intermediate section.

2. The vertebral rod of claim 1 wherein the intermediate section has at least one elongated recess within the interior surface and the outer surface has at least one elongated protrusion extending therealong, the at least one protrusion being disposed in the at least one recess.

3. The vertebral rod of claim 2 wherein the intermediate section includes a first recess within the interior surface disposed adjacent the first section and a second recess within the interior surface disposed adjacent the second section.

4. The vertebral rod of claim 2, wherein the resistance member includes a first elongated protrusion extending from the outer surface and a second elongated protrusion extending from the outer surface.

5. The vertebral rod of claim 2 wherein the at least one recess comprises an arcuate surface.

6. The vertebral rod of claim 2, wherein the at least one protrusion comprises an arcuate surface.

7. The vertebral rod of claim 1 wherein the open end is defined by opposing planar surfaces of the intermediate section.

8. The vertebral rod of claim 7 wherein the resistance member includes opposing planar surfaces configured for engagement with the planar surfaces of the intermediate section.

9. The vertebral rod of claim 1 wherein the cavity of the intermediate section defines a first distance and a second distance adjacent the open end, the first distance being greater than the second distance.

10. The vertebral rod of claim 1 wherein the resistance member extends into the open end to prevent engagement of the surfaces of the intermediate section defining the open end.

11. The vertebral rod of claim 1 wherein the resistance member extends beyond the cavity of the intermediate section.

12. The vertebral rod of claim 1 wherein the resistance member is monolithic.

13. The vertebral rod of claim 1 wherein the resistance member is elastic.

14. The vertebral rod of claim 1 wherein the resistance member is fabricated from thermoplastic or thermoset homopolymer or copolymer material.

15. A vertebral rod comprising
an elongated first section;
an elongated second section;
an intermediate section disposed between the first section and the second section, the intermediate section having an at least partially circumferential interior surface defining a cavity having an open end, the intermediate section including a first elongated recess within the interior surface disposed adjacent the first section and a second opposing elongated recess within the interior recess disposed adjacent the second section; and
a resistance member disposed in the cavity of the intermediate section and having an at least partially circumferential outer surface, the resistance member including a first elongated protrusion extending along the outer surface and a second elongated protrusion extending along the outer surface, the first protrusion disposed in the first recess and the second protrusion disposed in the second recess such that the entire interior surface engages the entire outer surface.

16. The vertebral rod of claim 15 wherein the open end is defined by opposing planar surfaces of the intermediate section.

17. The vertebral rod of claim 16 wherein the resistance member includes opposing planar surfaces configured for engagement with the planar surfaces of the intermediate section.

18. The vertebral rod of claim 15 wherein the cavity of the intermediate section defines a first distance and a second distance adjacent the open end, the first distance being greater than the second distance.

19. The vertebral rod of claim 15 wherein the resistance member extends beyond the cavity of the intermediate section.

20. An implant system comprising:
a vertebral rod comprising a first section, a second section configured to extending along a plurality of vertebral levels, an intermediate section disposed between the first section and the second section, the intermediate section having an interior surface defining a cavity having an open end, the intermediate section further having at least one elongated recess within the interior surface, and a resistance member disposed in the cavity of the intermediate section, the resistance member including an outer surface having at least one elongated protrusion extending therealong, the at least one protrusion being disposed in the at least one recess such that the entire interior surface engages the outer surface;
a first bone fastener for attaching the first section to tissue; and
a second bone fastener for attaching the second section to tissue.
